(19) United States
(12) Patent Application Publication
(10) Pub. No.: US 2026/0355086 A1
(43) Pub. Date: Nov. 12, 2026

(54) HAIR CARE ACTIVE AGENT

(71) Applicant: GIVAUDAN SA, Vernier (CH)

(72) Inventors: Amandine SCANDOLERA, Reims (FR); Daniel AURIOL, Rogues sur Garonne (FR); Romain REYNAUD, Toulouse (FR)

(21) Appl. No.: 19/715,260

(22) Filed: Jun. 22, 2026

Related U.S. Application Data
(63) Continuation of application No. 17/284,271, filed on Oct. 29, 2019, now abandoned.

(30) Foreign Application Priority Data
Oct. 29, 2018 (GB) 1817625.5

(51) Int. Cl. A61K 8/60; A61K 8/44; A61K 31/198; A61K 31/7048; A61Q 5/10

(52) U.S. Cl. CPC A61K 8/602; A61K 8/44; A61K 31/198; A61K 31/7048; A61Q 5/10; A61K 2800/522; A61K 2800/591

(57) ABSTRACT

The present invention provides a hair care active agent comprising taxifolin glucoside and N-acetyl tyrosine and a hair care composition comprising said hair care active agent. The hair care agent is able to reduce the occurrence of grey and white hair in an individual.

The present invention relates to a hair care active agent comprising taxifolin glucoside and N-acetyl tyrosine, to hair care compositions comprising the same, and its use and applications.

The loss of pigmentation in the hair shaft, leading to hair greying or canities, is one of the most obvious signs of aging. It is also one of the main beauty concerns appearing with age. Indeed, between age 45 and 65, about 74% of the worldwide population is affected by grey hair. The loss of pigmentation typically appears progressively between age 35 and 45. Despite affecting men and women, men seem to show a higher number of grey hairs. Hair greying differs with geographical and ethnic origins, with lower incidence in Asian and African populations rather than in Caucasians.

A human hair is formed of three different layers: the cuticle (external shell), the cortex (internal part containing melanin granules; responsible for hair color), and the medulla (soft core only present in mature white hair). The color of human hair is determined by its structure and the type and percentage of melanin it contains.

Melanin production in the hair is controlled by melanocytes located in the hair bulb matrix. Melanocyte activity is regulated by the normal hair cycle: During the anagen (growing) phase, the melanin produced by active melanocytes is transferred into cortical keratinocytes resulting in pigmentation of the entire hair shaft. During the catagen phase, the melanocytes enter apoptosis, and they disappear during the telogen phase. In order to produce a pigmented hair during the new anagen phase, a new pool of melanocytes migrates and differentiates from the hair follicle stem cells reservoir, containing in particular the outer root sheath cells (ORSc), to the hair bulb to naturally pigment the new hair.

Hair greying is explained by age-related functional changes in the stimulation and migration of the stem cells from the ORSc, but also by environmental factors. Indeed, the accumulation of reactive oxygen species (ROS) in melanocytes upon aging leads to the accumulation of mutations, decrease of antioxidant protection, inflammation, hair loss, and greying. ROS generated in scalp decrease the differentiation of melanoblasts into melanocytes, resulting in a lack of mature melanocytes in the bulb region. ROS also decrease the melanogenesis at bulb level, resulting in a decrease of melanin pigments transferred to the cortical keratinocytes.

Three types of hair can be identified when pigmentation loss starts to occur: colored hair (high content in melanin), grey hair (low melanocytes number and diluted content of melanin), and white hair (lacking melanocytes completely).

In an aging society, anti-aging products alleviating the signs of age are becoming more and more sought-after. For this reason, many people chose to color their grey or white hair. However, chemical hair colors are often aggressive and damaging to the hair. Moreover, users frequently complain about a lack of tolerance (itching, burning, prickling) and sustainability (regular recoloration necessary).

To avoid these drawbacks, it is desirable to find a way of positively influencing the natural pigmentation process of the hair.

To this end, US 2012/0114583 describes a hair treatment agent comprising (a) dihydroquercetin and/or a dihydroquercetin derivative; and (b) at least one amino acid. A preferred embodiment includes a six amino acid mixture of taurine, proline, valine, arginine, lysine, and glycine.

Dihydroquercetin (DHQ), or taxifolin, is a flavonoid found in certain conifers. It is an antioxidant and a stimulator of stem cells proliferation and maintenance. Taxifolin has previously been used in cosmetics for different purposes, including skin whitening (e.g. US 2011/0038968) and stimulation of pigmentation.

Furthermore, taxifolin is known to inhibit melanogenesis (Sang Mi An et al. Phytother. Res. 22, 1200-1207 (2008)).

It is an object of the present invention to provide a highly efficient hair care active agent for reducing the occurrence of grey and white hair in an individual and for restoring the natural hair color.

This object is achieved by the hair care active agent of the present invention and the hair care compositions comprising the same.

In a first aspect, the present invention provides a hair care active agent comprising taxifolin glucoside and N-acetyl tyrosine.

In a second aspect, the present invention provides a hair care composition comprising the hair care active agent of the present invention and a suitable carrier.

In a third aspect, the present invention refers to the non-therapeutic use of the hair care active agent of the present invention for stimulating the proliferation of hair follicle stem cells, for stimulating the active melanocytes production, for stimulating the melanogenesis, for stimulating the recovery of hair pigmentation, for reactivating the melanin production in grey hair, for activating the antioxidant defenses in hair follicles, for protecting the melanocytes against oxidative stress, for hair repigmentation, and/or for reducing the proportion and/or density of white or grey hair.

In a fourth aspect, the present invention provides a method of stimulating the proliferation of hair follicle stem cells, stimulating the active melanocytes production, stimulating the melanogenesis, stimulating the recovery of hair pigmentation, reactivating the melanin production in grey hair, activating the antioxidant defenses in hair follicles, protecting the melanocytes against oxidative stress, hair repigmentation, and/or reducing the proportion and/or density of white or grey hair, said method comprising the step of topically applying the hair care active agent of the present invention to human hair.

The combination of taxifolin glucoside and N-acetyl tyrosine provides an optimal anti-aging activity for hair by stimulating the melanin synthesis and thereby enhancing the natural coloring of the hair. It was found that within four months, the hair care active agent of the present invention significantly reduces the proportion and density of white and grey hair (up to 50% less grey hair).

The mode of action is completely independent from gender, hair type or hair color, making it an ideal solution to universally target hair greying.

The effects of the hair care active agent and the hair care composition of the present invention were studied in several in vitro and in vivo studies (see examples below).

The hair care active agent of the present invention was found to stimulate the stem cells proliferation, to stimulate the melanogenesis in hair follicles, to activate the antioxidant defenses in hair follicles, to protect the melanocytes against oxidative stress, to reactivate the melanin production in grey hair, to decrease the density of grey/white hair, and to repigment the hair. It is therefore particularly useful as an anti hair aging and anti grey and anti white hair agent.

While the melanogenesis stimulation of taxifolin and/or taxifolin derivatives in combination with at least one amino acid has been described before (US 2012/0114583), it was found that the hair care active agent of the present invention was even more effective.

Surprisingly, it has been found that the hair care active agent of the present invention is able to significantly stimulate the melanin synthesis in melanocytes, whereas taxifolin glucoside alone does not have any influence and a mixture of taxifolin glucoside and L-tyrosine causes only a slight and non-significant increase (see example 7 below).

Furthermore, the hair care active agent of the present invention also showed improved solubility.

In a preferred embodiment, at least part of the taxifolin glucoside is taxifolin alpha-D-glucoside. A suitable method of preparing taxifolin alpha-D-glucoside is described in WO 2007/144368, for instance.

In the hair care active agent of the present invention, taxifolin glucoside and N-acetyl tyrosine may be used alone or in combination with other active ingredients, adjuvants and/or solvents suitable for use in cosmetics.

In a preferred embodiment, the hair care active agent of the present invention further comprises taxifolin. Surprisingly, it was found that the solubility of taxifolin is improved in the presence of taxifolin glucoside: the solubility of taxifolin in water is typically about 1 g/L, but can be increased to 20 g/L or more in the presence of suitable amounts of taxifolin glucoside. The solubility of taxifolin in water is higher the higher the concentration of taxifolin glucoside in the aqueous solution, typically having a molar ratio of about 1:1. This allows for preparation of a more concentrated and therefore also more active formulation. Furthermore, thanks to the presence of taxifolin, the initial activity of the hair care active agent is increased.

A mixture of taxifolin and taxifolin glucoside may be prepared, for instance, by stopping the reaction according to WO 2007/144368 prior to full conversion, e.g. after about half of the taxifolin has been converted to obtain a roughly 1:1 mixture.

Taxifolin glucoside and taxifolin may be present in a weight ratio of from 100:0 to about 40:60. Preferably the weight ratio of taxifolin glucoside to taxifolin is from 90:10 to 40:60, more preferably from 70:30 to 50:50, and most preferably about 60:40.

In a preferred embodiment, the hair care active agent of the present invention further comprises glycine and/or epigallocatechin gallate and/or epigallocatechin gallatyl glucoside.

Glycine is the simplest possible amino acid. Glycine is typically used in cosmetics as a buffering agent. Furthermore, glycine is a collagen precursor.

Epigallocatechin gallate (EGCG, also known as epigallocatechin-3-gallate) is the ester of epigallocatechin and gallic acid, and is a type of catechin. Epigallocatechin gallate is known to stimulate hair growth. A suitable method of preparing epigallocatechin gallatyl alpha-D-glucoside is described in WO 2007/144368, for instance.

The hair care active agent of the present invention may further comprise other beneficial active agents, such as, for example, zinc chloride. Zinc is an enzyme cofactor that favors hair growth.

In the hair care active agent of the present invention, taxifolin glucoside and N-acetyl tyrosine may be used in any suitable concentration range and ratio. In particular, it is desirable to adapt the concentration ranges in order to guarantee sufficient activity and solubility.

The concentration of taxifolin glucoside in the hair care active agent of the present invention may be, for instance, from 0.01 to 0.50 wt %, more preferably from 0.05 to 0.25 wt %, and most preferably from 0.7 to 0.15 wt %, for example about 0.10 wt %.

The concentration of N-acetyl tyrosine in the hair care active agent of the present invention May be, for instance, from 1.0 to 30 wt %, more preferably from 10 to 20 wt %, and most preferably from 13.5 to 16.5 wt %, for example about 15.0 wt %.

In the hair care active agent of the present invention, the weight ratio of taxifolin glucoside to N-acetyl tyrosine is preferably from 1:5,100 to 1:3.4, more preferably from 1:340 to 1:70, and most preferably from 1:200 to 1:100, for example about 1:150.

Also all the other active ingredients, adjuvants and/or solvents may be used in the hair care active agent of the present invention in any suitable concentration range and ratio. In particular, it is desirable to adapt the concentration ranges in order to guarantee sufficient activity, solubility, stability, and ease of formulation.

For instance, glycine may be used in a concentration of from 0.01 to 0.50 wt %, more preferably from 0.10 to 0.30 wt %, and most preferably from 0.12 to 0.18 wt %, for example about 0.15 wt %.

For instance, epigallocatechin gallatyl glucoside may be used in a concentration of from 0.001 to 0.60 wt %, more preferably from 0.010 to 0.060 wt %, and most preferably from 0.015 to 0.045 wt %, for example about 0.03 wt %.

For instance, sodium metabisulfite may be used in a concentration of from 0.01 to 1.00 wt %, more preferably from 0.10 to 0.750 wt %, and most preferably from 0.45 to 0.55 wt %, for example about 0.50 wt %. Sodium metabisulfite prevents the oxidation of polyphenols.

In a preferred embodiment, the hair care active agent of the present invention comprises 13.5 to 16.5 wt % of N-acetyl tyrosine, 0.12 to 0.18 wt % of glycine, 0.05 to 0.09 wt % of zinc chloride, 0.08 to 0.12 wt % of taxifolin glucoside, 0.5 to 0.08 wt % of taxifolin, 0.015 to 0.045 wt % of epigallocatechin gallatyl glucoside, 0.45 to 0.55 wt % of sodium metabisulfite, and 47.5 to 52.5 wt % of glycerol.

In a particularly preferred embodiment, the hair care active agent of the present invention comprises about 15.0 wt % of N-acetyl tyrosine, about 0.15 wt % of glycine, about 0.07 wt % of zinc chloride, about 0.10 wt % of taxifolin glucoside, about 0.07 wt % of taxifolin, about 0.03 wt % of epigallocatechin gallatyl glucoside, about 0.50 wt % of sodium metabisulfite, and about 50.0 wt % of glycerol.

In the above hair care active agents of the present invention, part of the taxifolin glucoside may be replaced by taxifolin. In particular, up to 60% of taxifolin glucoside may be replaced by taxifolin, preferably up to 50%, and even more preferably up to 40%.

In a preferred embodiment, the hair care active agent of the present invention comprises 13.5 to 16.5 wt % of N-acetyl tyrosine, 0.12 to 0.18 wt % of glycine, 0.05 to 0.09 wt % of zinc chloride, 0.14 to 0.20 wt % of a 60:40 mixture of taxifolin glucoside and taxifolin, 0.015 to 0.045 wt % of epigallocatechin gallatyl glucoside, 0.45 to 0.55 wt % of sodium metabisulfite, and 47.5 to 52.5 wt % of glycerol.

In a particularly preferred embodiment, the hair care active agent of the present invention comprises about 15.0 wt % of N-acetyl tyrosine, about 0.15 wt % of glycine, about 0.07 wt % of zinc chloride, about 0.17 wt % of a 60:40 mixture of taxifolin glucoside and taxifolin, about 0.03 wt % of epigallocatechin gallatyl glucoside, about 0.50 wt % of sodium metabisulfite, and about 50.0 wt % of glycerol.

Alternatively, taxifolin may be added to the above hair care active agents of the present invention in addition to the indicated amounts of taxifolin glucoside. In this case, taxifolin may be added in an amount of up to 0.24 wt %, more preferably of 0.07 to 0.20 wt %, and most preferably of 0.10 to 0.15 wt %. Preferably, the weight ratio of taxifolin to taxifolin glucoside is 1.5:1 or lower.

In a further aspect, the present invention also provides a hair care composition comprising the hair care active agent described above and a suitable carrier.

“Hair”, as used herein, means human hair including scalp hair, facial hair, and body hair, particularly hair on the human head and scalp, including eye brows, beard and moustache.

The term “hair care composition” includes both leave-on products and wash-out, such as shampoos, sprays, lotions, etc.

For example, there are hair cleansing compositions, hair conditioning compositions, and hair styling compositions, such as shampoos, conditioners, sprays, treatments, masks, strengtheners, pre-shampoos, lotions, serums, creams, foams, mousses, and gels. Specific examples include, but are not limited to, anti grey hair lotions, anti white hair shampoos, natural repigmenting hair masks, anti grey hair for beard and mustache, hair color recovery sprays, and premature grey/white hair treatment gels. Many of these compositions that are known are water-based formulations.

Hair cleansing compositions are generally effective to remove soil from hair. The soil includes natural exudations from the scalp, environmental agents, and styling products. The soil can coat or deposit on the hair and scalp. Hair coated with such soil is typically greasy in feel and appearance, heavy to the touch, possibly malodorous, and generally unable to maintain a desired style. Known cleansing compositions typically include a combination of water and surface-active ingredients, such as soap or synthetic surfactants, and may also include a non-aqueous blend of starches. The combination of water and surface-active agents emulsifies the soil from the hair and scalp, allowing it to be rinsed away.

Cleansing compositions may also contain conditioning agents that deposit on the hair and scalp during rinsing with water. Such conditioning agents can include polymers, oils, waxes, protein hydrolysates, silicones, and mixtures and derivatives thereof. In addition, the conditioning composition can be a separate and different product from the cleansing composition.

Conditioning compositions that are known in the art are typically water-based formulations. However, there are also known conditioning compositions, which include at least one of silicones; animal, mineral or vegetable oils; waxes; petrolatums; and greases. The water-based conditioning compositions typically include substituted cationic waxes, fatty alcohols, cationic polymers, hydrolyzed proteins and derivatives thereof, and fragrances. Such conditioning formulations impart combability and manageability to the treated hair, thereby minimizing breakage during the styling process and resulting in shiny, healthy, and manageable hair. Conditioning compositions may also be effective to moisturize the hair. Subsequent drying and styling processes can include air drying or heating.

The suitable carrier must be cosmetically acceptable.

“Cosmetically acceptable”, as used herein, means that the carrier is suitable for use in contact with human keratinous tissue without undue toxicity, incompatibility, instability, allergic response, and the like. All compositions described herein, which have the purpose of being directly applied to keratinous tissue, are limited to those being cosmetically acceptable.

Hair care compositions typically comprise the carrier at a level from about 20 wt % to about 99 wt %. The carrier may comprise water, organic solvents (miscible or non-miscible with water) silicone solvents, and/or mixtures thereof. The solvents should be dermatologically acceptable. Carriers usually do not comprises more than about 2 wt % of non-volatile solvent, as significantly higher concentrations will increase hair weigh-down and greasy feel. Water, organic and silicone solvents that have boiling points below or equal to 250 °C are considered volatile solvents. Suitable carriers typically include water and water solutions of lower alkyl alcohols, such as monohydric alcohols having 1 to 6 carbons (e.g. ethanol and/or isopropanol), and polyhydric alcohols, such as glycols, glycerine, and other diols.

The hair care composition according to the present invention may further comprise one or more materials selected from the group consisting of solvents, surfactants, thickeners, styling polymers, anti-dandruff actives, antimicrobial materials, skin and scalp actives, vitamins, salts, buffers, hair growth agents, conditioning materials, hair-fixative polymers, fragrances, colorings/colorants, dyes, pigments, opacifiers, pearlescent aids, oils, waxes, preservatives, sensates, sunscreens, medicinal agents, antifoaming agents, antioxidants, binders, biological additives, buffering agents, bulking agents, chelating agents, chemical additives, film formers or materials, pH adjusters, propellants, oxidizing agents, and reducing agents.

All additives should be physically and chemically compatible with the essential components of the hair care composition, and should not otherwise unduly impair stability, aesthetics or performance. Most importantly, they should also be cosmetically acceptable.

As thickeners, the hair care composition may comprise rheology modifiers to improve feel, in-use properties and suspending stability. For example, rheological properties may be adjusted so that the hair care composition remains uniform during its storage and transportation and does not drip undesirably onto other areas of the body, clothing or home furnishings during its use. Any suitable rheology modifier can be used. Typically, about 0.01 to about 3 wt % of thickener is included. Examples of suitable thickeners are disclosed in WO 2015/035164 and US 2001/0043912.

The hair care composition of the present invention can additionally also comprise any suitable optional ingredients as desired. Such optional ingredients should be physically and chemically compatible with the components of the hair care composition, and should not otherwise unduly impair stability, aesthetics or performance. The CTFA Cosmetic Ingredient Handbook, Tenth Edition (published by the Cosmetic, Toiletry, and Fragrance Association, Inc., Washington D.C.) (2004) describes a wide variety of non-limiting materials that can be added to the hair care composition of the present invention.

For instance, the hair care composition of the present invention can include a styling polymer. A styling polymer may be selected from the group consisting of acrylate polymers and their esters, methacrylate polymers and their esters, acrylate copolymers and their esters, methacrylate copolymers and their esters, polyurethane polymers and copolymers, polyvinylpyrrolidones (PVP), PVT-polyvinyl acetate copolymers, PVP-polyvinyl alcohol copolymers, polyesters, and other polymers.

The hair care composition of the present invention may also comprise a sensate. As used herein, the term “sensate” refers to a substance that, when applied to the skin, causes a perceived sensation of a change in conditions, for example but not limited to heating, cooling, refreshing, and the like. Sensates are preferably utilized at levels from about 0.001 to about 10 wt % of the consumer product. Examples of suitable sensates include camphor, menthol, L-isopulegol, ethyl menthane carboxamide, and trimethyl isopropyl butanamide.

The hair care composition of the present invention may also contain optional components, which modify the physical and performance characteristics. Such components include surfactants, salts, buffers, thickeners, solvents, opacifiers, pearlescent aids, preservatives, fragrances, colorants, dyes, pigments, chelators, sunscreens, vitamins, and medicinal agents. Optional components that are among those useful herein are disclosed in U.S. Pat. No. 4,387,090.

The hair care composition of the present invention may also optionally contain an anti-dandruff agent for providing anti-microbial activity. The anti-dandruff agent may be particulate or soluble. Preferred anti-dandruff agents include, but are not limited to, particulate crystalline anti-dandruff agents, such as sulfur, selenium sulphide, and heavy metal salts of pyridinethione. Especially preferred is zinc pyridinethione. Soluble anti-dandruff agents, such as ketoconazole, are also known in the art. An anti-dandruff agent is preferably present in a concentration of about 0.1 to 4 wt %.

The hair care composition of the present invention may also optionally contain hair growth agents, such as zinc pyridinethione. The compositions and consumer products of the present invention may also optionally contain a compound useful for regulating the growth and loss of hair. Such compounds known in the art include lupine triterpenes and derivatives thereof, derivatives of oleanane triterpenes and ursane triterpenes, and salts and mixtures thereof, minoxidil (6-(1-piperidinyl)-2,4-pyrimidinediamine 3-oxide), or finasteride.

The hair care composition of the present invention may also optionally contain salts and/or buffers in order to modify the rheology. For example, salts, such as potassium chloride and sodium chloride, may be added at levels from about 0.001 to about 1 wt %. Buffers, such as citrate or phosphate buffers, may also be used. Preferably, the pH of the present consumer products are modified to a pH from about 3 to about 10, preferably from about 3 to about 7.

The hair care composition of the present invention may also optionally contain additional conditioning polymers, in particular cationic conditioning polymers. If present, these are preferably employed at a level of from about 0.5 to about 10 wt %. Suitable cationic conditioning polymers are disclosed in US 2001/0043912.

A wide variety of other additional components can be formulated into the present hair care composition. These include: other conditioning agents, such as hydrolyzed collagen, vitamin E, panthenol, panthenyl ethyl ether, hydrolyzed keratin, proteins, plant extracts, and nutrients; hair-fixative polymers, such as amphoteric, non-ionic, cationic, and anionic fixative polymers, and silicone grafted copolymers; preservatives, such as benzyl alcohol, methyl paraben, propyl paraben, and imidazolidinyl urea; pH adjusting agents, such as glutamic acid, citric acid, sodium citrate, succinic acid, phosphoric acid, lactic acid, sodium hydroxide, and sodium carbonate; salts in general, such as potassium acetate and sodium chloride; coloring agents; hair oxidizing (bleaching) agents, such as hydrogen peroxide, perborate, and persulfate salts; hair reducing agents, such as thioglycolates; fragrances; and sequestering agents, such as disodium ethylenediamine tetra-acetate; ultraviolet and infrared screening and absorbing agents, such as octyl salicylate; and mixtures thereof.

Additional optional ingredients include, but are not limited to: skin and scalp actives, oils, waxes, antifoaming agents, antioxidants, binders, biological additives, bulking agents, chelating agents, chemical additives, film formers or materials, and propellants.

In a further aspect, the present invention relates to the non-therapeutic use of the hair care active agent of the present invention for stimulating the proliferation of hair follicle stem cells, for stimulating the active melanocytes production, for stimulating the melanogenesis, for stimulating the recovery of hair pigmentation, for reactivating the melanin production in grey hair, for activating the antioxidant defenses in hair follicles, for protecting the melanocytes against oxidative stress, for hair repigmentation, and/or for reducing the proportion and/or density of white or grey hair.

In a further aspect, the present invention relates to a method of stimulating the proliferation of hair follicle stem cells, stimulating the active melanocytes production, stimulating the melanogenesis, stimulating the recovery of hair pigmentation, reactivating the melanin production in grey hair, activating the antioxidant defenses in hair follicles, protecting the melanocytes against oxidative stress, hair repigmentation, and/or reducing the proportion and/or density of white or grey hair, said method comprising the step of topically applying the hair care active agent according to the present invention.

The hair care active agent of the present invention may be applied to either wet or dry hair, depending on formulation.

Preferably, the hair care active agent of the present invention is applied in the form of the hair care composition of the present invention described above.

The above beneficial effects of the hair care active agent of the present invention have been confirmed by extensive in vitro, ex vivo, and clinical studies, some of which are described in the examples below.

This patent application contains the following figures:

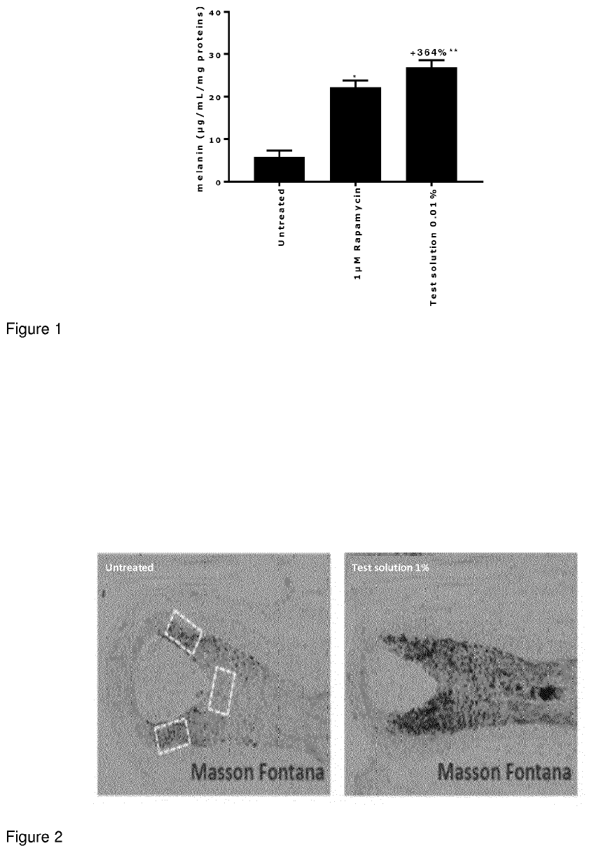

FIG. 1: Melanin content produced by the co-culture between Normal Human Melanocytes (NHMs) and Normal Human Epidermal Keratinocytes (NHEKs) after 72 h of incubation in absence and in presence of the test solution from Example 1 in 1:1 NHM/NHK medium at 0.01%. Student t-test ** p-value <0.01 and *p-value <0.05.

FIG. 2: Evaluation of melanin content through Fontana Masson staining of gray micro-dissected hair follicles treated with the standard solution from Example 1 at 1%.

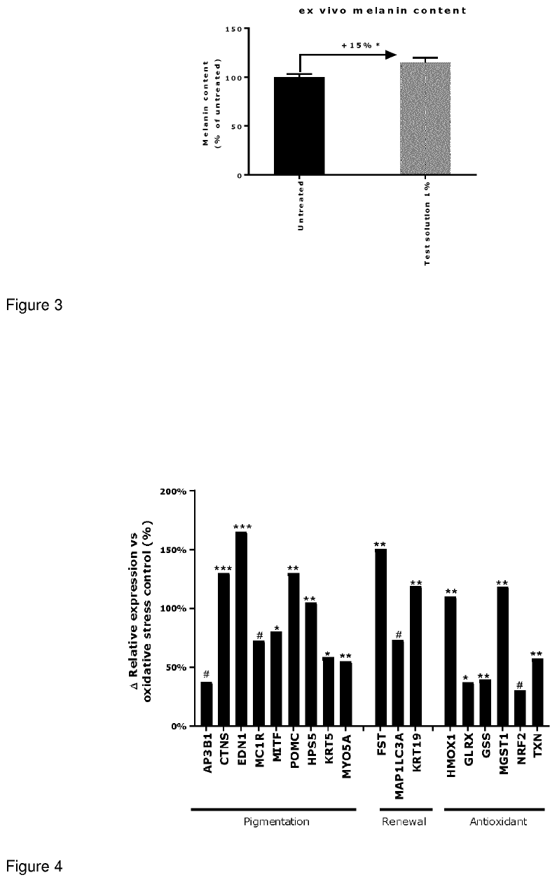

FIG. 3: Evaluation of melanin content through Fontana Masson staining of gray micro-dissected hair follicles treated with the standard solution from Example 1 at 1%. Student t-test *p-value <0.05.

FIG. 4: Gene expression analysis after induction of an oxidative stress in full ex vivo scalp. ANOVA with Fisher's LSD test ** *p-value <0.01, *p-value <0.05 and #p-value <0.1.

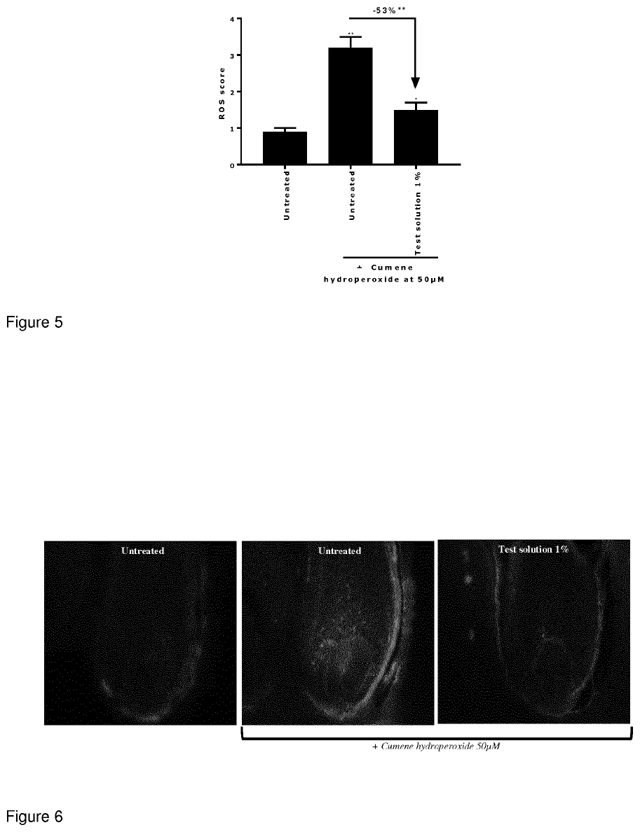

FIG. 5: Evaluation of ROS production after 1 h of the standard solution from Example 1 pre-treatment then 1 h with cumene hydroperoxide at 50 μM. One-way ANOVA with permutation test, F (2,33) 31.33; * p-value <0.05 and ** p-value <0.01, followed by t-test with permutation.

FIG. 6: Representative pictures of the ROS production analysis in micro-dissected hair follicles after an induction by cumene hydroperoxide at 50 μM with or without treatment with the standard solution from Example 1 at 1%.

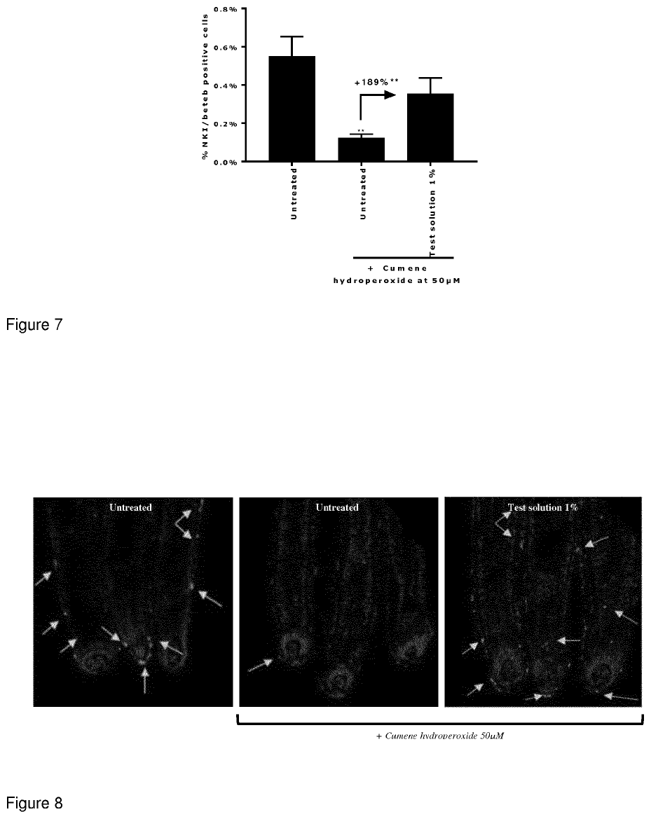

FIG. 7: Percentage of NKI/beteb positive cells after an oxidative stress and followed by treatment with the standard solution from Example 1 at 1%. One-way ANOVA with permutation test, F(2,33) 7.91; ** p-value <0.01, followed by t-test with permutation.

FIG. 8: Representative pictures of NKI/beteb (red) immunostaining representing HFs melanocytes and melanoblasts with DAPI counterstaining (blue). The different conditions presented are the hair follicles micro-dissected after an induction by cumene hydroperoxide at 50 μM with or without treatment with the standard solution from Example 1 at 1%.

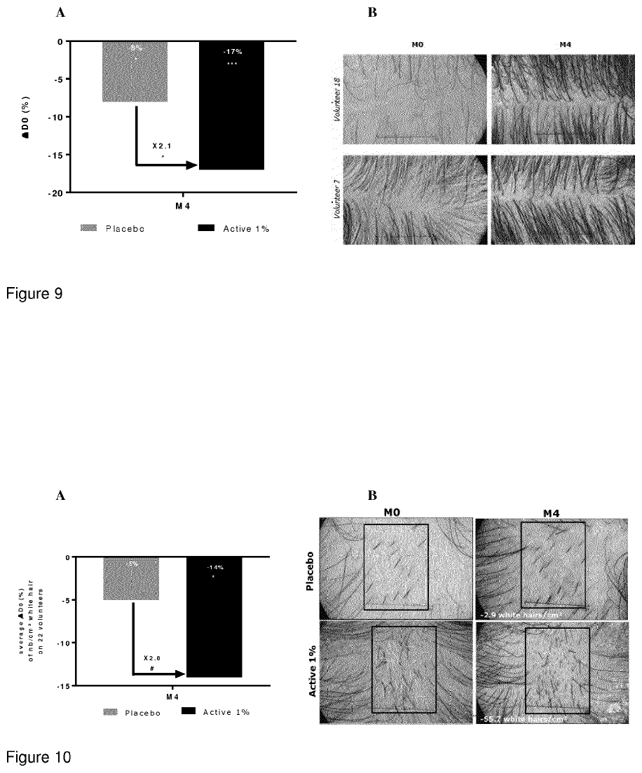

FIG. 9: A. Scoring of the proportion of white hair (T4M in %)
B. Representative images of the hair lotion containing the active ingredient of the present invention at 1% on the reduction of the proportion of white hair

FIG. 10: A. Scoring of the density of white hair (T4M)
B. Representative pictures of the reduction of the density of white hair

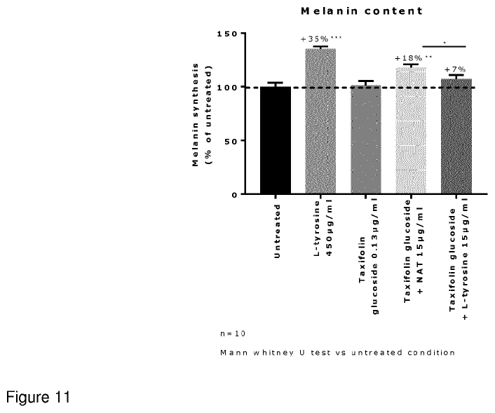

FIG. 11: Melanin content in % of the untreated condition

The present invention is thus further illustrated by means of the following non-limiting examples:

### EXAMPLE 1: HAIR CARE ACTIVE AGENT ACCORDING TO THE PRESENT INVENTION

### Standard Solution

The following standard solution may be used to prepare a hair care active agent of the present invention:

Standard Solution

| Substance | Concentration |
|---|---|
| N-acetyl tyrosine | 15.0 wt % |
| glycine | 0.15 wt % |
| zinc chloride | 0.07 wt % |
| taxifolin glucoside | 0.17 wt % |
| epigallocatechin gallatyl glucoside | 0.03 wt % |
| sodium metabisulfite | 0.50 wt % |
| glycerol | 50.0 wt % |

The hair care active agent of the present invention may consist of said standard solution, or may comprise said standard solution in combination with other ingredients.

Thus, the standard solution may be used as such.

Alternatively, the standard solution may also be diluted in a solvent, buffer or culture medium.

### Test Solution

For some of the examples below (see indications in examples), a test solution was prepared by diluting the above standard solution in William's E medium to a final concentration of 0.01 to 1% v/v.

### INCI

A hair care composition comprising the hair care active agent of the present invention may be described by the following INCI (International Nomenclature of Cosmetic Ingredients) indications:
WATER, GLYCERIN/ACETYL TYROSINE/SODIUM METABISULFITE/LARIX EUROPAE WOOD EXTRACT/GLYCINE/ZINC CHLORIDE/EPIGALLOCATECHIN GALLATYL GLUCOSIDE, SODIUM BENZOATE, CITRIC ACID, SODIUM CITRATE, PPG-26 BUTETH-26, PEG-40 HYDROGENATED CASTOR OIL, AQUA/WATER, FRAGRANCE, BUTYLPHENYL, METHYLPROPIONAL, D-LIMONENE, ALPHA-ISOMETHYLIONONE.

### EXAMPLE 2: EVALUATION OF MELANIN PRODUCTION: IN VITRO CO-CULTURE MODEL

### Culture and Experimental Design

The cells used in this study were primary cultures of normal human keratinocytes (NHK) and normal human melanocytes (NHM) extracted after skin surgery (foreskin) of an 8 years old Caucasian male donor (phenotype III/IV).

The wells were plated with 200,000 NHK/9.6 cm² and 50,000 NHM/9.6 cm² and grown for 24 hours in supplemented medium (1:1 NHM supplemented medium/NHK supplemented medium). After 24 hours of culture, cells were treated (conditions: untreated; 0.01% of the test solution from Example 1 in a 1:1 mixture of NHM supplemented medium and NHK supplemented medium without supplements; and 1 μM Rapamycin) 3 times 24 hours.

### Colorimetric Assay of Melanin

Melanin content in cells pellets was performed in parallel with proteins assay.

The cell pellets were taken up in NaOH at 60 °C for 30 minutes. The concentration of melanin in supernatants and in standard range of melanin synthetic were determined by lecture at 405 nm, using synthetic melanin as control.

### Protein Assay (BCA)

Total proteins assay in cells pellets was performed in parallel by colorimetric method based on bicinchoninic acid. Standard range was prepared with BSA (Bovine Serum Albumin).

The cell pellets were taken up in NaOH at 60 °C for 30 minutes. Dosage was realized by adding a mixture of the reagents (bicinchoninic acid+CuSO₄) to aliquots of lysates (cells pellets lysed). The plates were incubated at 37 °C for 30 minutes and then lecture is performed at 570 nm.

### Expression of Results

For both melanin and protein dosages, raw data, i.e. OD measurements obtained for respective standards were plotted on graphics to obtain standard calibration curves. Then, the amount/concentrations of proteins or melanin measured in the samples were determined.

Quantitative values of each condition were averaged. Data were graphically presented as amount/concentrations (μg/mL). Results obtained for each condition was also expressed relatively to the untreated control, set to 100%:

%_{«sample»}=(Mean OD_{«sample»}/Mean OD_{«control»})×100

To obtain melanin concentrations reported to the total proteins amount (μg/mL/mg of proteins), each concentration value (μg/mL) was divided by each proteins data (mg), well by well. Values of each condition were then averaged.

The statistically significant effects of the results were determined by the Student's t-test.

### Results

After 72 hours of treatment, the positive reference (1 μM Rapamycin) was found to stimulate significantly the production of melanin in the co-culture model (+282%*) confirming that the model was robust and respondent (FIG. 1).

The presence of the test solution from Example 1 at 0.01% also demonstrated a stimulating effect on the melanin production. Indeed, the quantity of melanin was increased by 363%**.

### EXAMPLE 3: EVALUATION OF MELANIN PRODUCTION: EX VIVO GREY HAIR FOLLICLES

### Tissue Samples

Micro-dissected hair follicles (HFs) were obtained from occipital healthy human follicular units skin obtained from a healthy female donor (35 years old; donor 1) undergoing hair transplantation surgery or from scalp biopsy from a male donor (53 years old; donor 2) after informed consent and ethics approval (University of Muenster, n.2015-602-f-S).

### Hair Follicles Organ Culture

Microdissected human anagen VI scalp HFs (60 and 26 HFs/experiment, respectively) were cultured at 37 °C with 5% CO₂ in a minimal media of William's E media (Gibco, Life Technologies) supplemented with 2 mM of L-glutamine (Gibco), 10 ng/ml hydrocortisone (Sigma-Aldrich), 10 μg/mL insulin (Sigma-Aldrich) and 1% penicillin/streptomycin mix (Gibco) to make Williams Complete Media (WCM; J Cell Sci. 1990 November; 97 (Pt 3):463-71. Human hair growth in vitro. Philpott MP1, Green M R, Kealey T; Exp Dermatol. 2010 March; 19(3):305-12. Methods in hair research: how to objectively distinguish between anagen and catagen in human hair follicle organ culture. Kloepper J E1, Sugawara K, Al-Nuaimi Y, Gáspár E, van Beek N, Paus R; Exp Dermatol. 2015 December; 24(12):903-11. Human hair follicle organ culture: theory, application and perspectives. Langan E A, Philpott M P, Kloepper J E, Paus R). After 24 hrs, the medium was replaced with fresh medium containing either WCM (vehicle) or the Test Solution at final concentrations of 1% diluted in WCM. 11 ‘grey’ (defined by being low pigmented) anagen VI HFs were cultured per experimental group. HFs were cultured for a total of 3 days.

### Frozen Hair Follicles Processing

Frozen samples were sectioned with a cryostat (CM3050S, Leica Biosystems) and 6 μm sections were collected. The HFs were carefully orientated to obtain intact hair follicle sections and open dermal papillae. Consecutive sections of hair follicles were collected and slides were stored at −80 °C.

### Masson Fontana Histochemical Staining

To evaluate melanin as a marker for HF pigmentation, Masson Fontana (MF) staining was performed as previously described (Exp Dermatol. 2010 March; 19(3):305-12. Methods in hair research: how to objectively distinguish between anagen and catagen in human hair follicle organ culture. Kloepper JE1, Sugawara K, Al-Nuaimi Y, Gáspár E, van Beek N, Paus R) on frozen slides. In short, melanin was stained with silver nitrate (Caesar & Loretz, Hilden, Germany) ammonia-based solution and developed with 5% aqueous sodium thiosulphate (Merck Millipore, Darmstadt, Germany).

### Quantitative (Immuno-) Histomorphometry

Pictures were taken with a Keyence Biozero Microscope 8100 and 9000 at an original magnification of 200×.

### Melanin Content in Anagen VI Hair Follicles

Three areas of 100×175 or 113×159 pixel were measured above the Auber's line to assess pigmentation intensity for donor 1 or 2 in anagen HFs, respectively, using the ImageJ software (Rasband, W. S., ImageJ, U. S. National Institutes of Health, Bethesda, Maryland, USA, https://imagej.nih.gov/ij/).

### Data Management

All data were expressed as mean or fold change of Mean±SEM. Gaussian distribution was tested with Shapiro-Wilk normality test. Outlier analysis was performed by two-sided Grubbs' test of the pooled data for donor 1 and 2. Significant outliers were removed and subsequently statistical analysis was performed using Student's t test comparing the results of each tested group to vehicle using GraphPad Prism 6 (GraphPad Software). A p value of <0.05 was considered statistically significant (*).

### Results

The induction of melanin production was evaluated through Fontana Masson staining quantification.

As can be seen in FIGS. 2 and 3, the test solution from Example 1 was able to induce melanogenesis in greying hair follicles and stimulate the melanin production by 15% (p<0.05).

These results show that the hair care active agent of the present invention is not only able to stimulate melanogenesis in melanocytes, but also in greying hair follicles.

### EXAMPLE 4: TRANSCRIPTOMIC ANALYSIS IN A FULL EX VIVO SCALP

### Skin Explants Culture

The test was carried out on skin explants NativeSkin®, a full-thickness skin biopsy embedded in a solid and nourishing matrix while its epidermal surface is left in contact with air. The skin biopsy is firmly embedded in the matrix that prevents any lateral diffusion of topically applied formulations.

The study was conducted on lift explant from 3 donors with sufficient hair follicles and equivalent number between the samples from a same donor:
Donor 1: 66 years old Caucasian Woman with 18 follicles for the untreated, 19 follicles for oxidative treatment and 19 follicles for oxidative treatment with 1% of the test solution from Example 1 in Carbopol®.
Donor 2: 58 years old Caucasian Woman with 10 follicles for the untreated, 10 follicles for oxidative treatment and 9 follicles for oxidative treatment with 1% of the test solution from Example 1 in Carbopol®.
Donor 3: 64 years old Caucasian Woman with 30 follicles for the untreated, 30 follicles for oxidative treatment and 31 follicles for oxidative treatment with 1% of the test solution from Example 1 in Carbopol®.

Carbopol® consists of acrylic acid crosslinked with allyl sucrose or allyl pentaerythritol.

### Product Evaluation and Design of Study

The study was conducted on 3 donors (1 explant per condition).

Oxidative stress was applied for 3 days, once a day, by adding 9 mg of Hypoxanthin+10 units of Xantin oxidase during 1 h of reaction to produce free O₂^{·-} radicals at the skin surface.
Explant+oxidative stress+placebo (Carbopol®) for 48 h
Explant+oxidative stress+test solution from Example 1 at 1% for 48 h

After treatment, the dermis of each explant was removed by microsurgery to focus gene quantification on epidermal cells.

### Method of Analysis

qPCR microfluidic technology according to the protocol of Fluidigm® (BMC Genomics. 2011 Mar. 9; 12:144. Quantitative miRNA expression analysis using fluidigm microfluidics dynamic arrays. Jang J S1, Simon V A, Feddersen R M, Rakhshan F, Schultz D A, Zschunke M A, Lingle W L, Kolbert C P, Jen J) was used. Microfluidic technology comes from crossing the world of nanotechnology and gene analysis by q-PCR. System miniaturization has led to the development of a chip that currently allows analysing 48 conditions versus 48 genes.

The targeted genes cover the following functions:
Hair physiology
Antioxidant activities
Pigmentation

Skin explants were collected in specific lysis solution for mRNA extraction. Lysates were transferred on plate in order to purify mRNA. Afterwards, a reverse transcription system was used.

According to the Fluidigm® protocol, specific stages for 48×48 chip preparation were started. A pre-amplification step was carried out with the primers used in the chip. Pre-amplified cDNA/PCR mix and primers were deposited on the chip. The mixture blending was undertaken by the IFC Controller and then the chip was placed in the BioMark™ system in order to carry out real time PCR.

To confirm an activating or inhibitory effect, the values were compared to the control explant treated with Carbopol® only. The results are expressed as relative expression rate.

### Results

The impact of oxidative stress was investigated at the transcriptomic level. The scalps were repeatedly stimulated by a mixture of xanthin and hypoxanthin in order to mimic the oxidative stress experienced by the hair follicles during human life. Then, the Test Solution was applied for 48 hours as a curative treatment.

The results presented in FIG. 4 show the difference of the relative expression compared to the oxidative stress control.

It was found that the test solution from Example 1 induced a positive response on various biological functions important in the preservation of melanin content.

Indeed, the test solution from Example 1 was found to stimulate the expression of proteins involved in melanosome biogenesis and transport, such as AP3B1 (+37.8%#), CTNS (+130.4%***), HPS5 (+105.14**), KRT5 (+58.7%*) and MYO5A (+55.07%**).

It was also found to stimulate the expression of major genes involved in the regulation of the melanin synthesis, such as EDN1 (+165.56%***), MC1R (+72.64%#), MITF (+80.51%*) and POMC (+130.51%**).

Furthermore, the test solution from Example 1 had an impact on the expression of genes involved in renewal and autophagy, such as FST (150.95%**), KRT19 (+119.28%**) and MAP1LC3A (+73.47%#).

Finally, the test solution from Example 1 induced the expression of genes participating of the antioxidant response at various level such as HMOX1 (+110.47%**), GLRX (+37.47%*), GSS (+39.70%**), MGST1 (+118.63%**) and NRF2 (+30.41%#).

### EXAMPLE 5: EVALUATION OF THE IMPACT OF OXIDATIVE STRESS ON MICRO-DISSECTED HAIR FOLLICLES

### Microdissection and Hair Shaft Elongation

Anagen VI human hair follicles (HFs) were dissected from the scalp (Donor: female born in 1962). The HFs used were selected after 18 hours of pre-culture. The following parameters were evaluated in the selection procedure: growth rate (>0.18 mm/18 hours), morphology (no signs of dystrophy).

Starting from day 1, after the selection at day 0, the test compounds were dissolved in the culture medium and the HFs were cultured up to planned endpoints. Twelve hair follicles were used per treatment for each read out parameter. The culture medium was renewed every other day.

### ROS Evaluation

After the selection, the HFs were incubated for 1 hour with the standard solution from Example 1 dissolved in William's E medium. Afterwards, the HFs were cultured for 30 min in the presence of dichlorofluorescein diacetate (DCFH-DA), a probe that reacts with ROS to become fluorescent. Following the DCFH-DA incubation, the HFs were rinsed in PBS and incubated with cumene hydroperoxide at 50 μM (oxidative stimulus) for 1 hour. At the end of the experimental phase, the HFs were harvested, cryo-fixed and cut at the cryo-micro-tome for consequent image acquisition and image analysis of fluorescence within the sections. A slide for each HF was processed by image acquisition and related analysis (i.e. 12 images for each treatment).

### NKI/Beteb Evaluation

Total melanocytes quantification was obtained following NKI/beteb-DAPI double immunostaining of the cryo-sections. NKI/beteb antibody (#MON7006-1 by Monosan) recognizes a (pre-) melanosomal antigen present in all melanocytes (i.e. both active melanocytes and melanoblasts).

On the immunostained sections, image analysis was performed in order to quantify the number of NKI-beteb positive cells within each hair follicle. The obtained value was normalized relative to the total number of cells of the considered area.

### Image and Statistical Analysis

Image analysis was performed using ImageJ software (NIH, USA).

All quantitative data was summarized in terms of the mean score, standard deviation and standard error of the mean for each treatment.

Differences between groups were evaluated by One-way ANOVA with permutation test followed by Tukey and t-test with permutation.

Results: Limitation of Oxidative Stress by the Standard Solution from Example 1 in Micro-Dissected Hair Follicles

The oxidative stress represented by the treatment with 50 μM of cumene hydroperoxide induced a significant increase of ROS production (+256%** compared to the untreated condition). The pre-treatment with the standard solution from Example 1 reduced considerably the ROS production by 53%. Thus, the reduction was significant compared to the cumene peroxide control with p<0.01 showing a preventive effect of the active (FIG. 5).

Representative pictures are presented FIG. 6.

### Results: Impact of Oxidative Stress on Melanocytes in Micro-Dissected Hair Follicles

It was observed that cumene hydroperoxide significantly reduced the number of melanocytes by 78%** compared to the untreated condition. When treated with the standard solution from Example 1, the oxidative stress induced a slight decrease of melanocytes, but the presence of the active mitigated considerably its deleterious effect. Indeed, compared to the cumene hydroperoxide control, the standard solution from Example 1 increased the percentage of NKI/beteb positive cells by +189%**, confirming a major impact on hair follicles melanocytes and melanoblasts protection (FIG. 7).

Representative pictures are presented in FIG. 8.

### EXAMPLE 6: CLINICAL INVESTIGATION

### Introduction

Two clinical evaluation methods were performed in order to demonstrate the in vivo efficacy of a lotion containing the hair care active agent of the present invention versus a placebo lotion:
reduction of the proportion of white hair using the scoring method
reduction of the proportion of white hair using the counting method of white hair density (number of hair/cm²).

### Description of Compositions Used

Hair lotion containing 1% of the hair care active agent of the present invention:
WATER, GLYCERIN/ACETYL TYROSINE/SODIUM METABISULFITE/LARIX EUROPAE WOOD EXTRACT/GLYCINE/ZINC CHLORIDE/EPIGALLOCATECHIN GALLATYL GLUCOSIDE, SODIUM BENZOATE, CITRIC ACID, SODIUM CITRATE, PPG-26 BUTETH-26, PEG-40 HYDROGENATED CASTOR OIL, AQUA/WATER, FRAGRANCE, BUTYLPHENYL, METHYLPROPIONAL, D-LIMONENE, ALPHA-ISOMETHYLIONONE.

### Placebo Hair Lotion:

WATER, SODIUM BENZOATE, CITRIC ACID, SODIUM CITRATE, PPG-26 BUTETH-26, PEG-40 HYDROGENATED CASTOR OIL, AQUA/WATER, FRAGRANCE, BUTYLPHENYL, METHYLPROPIONAL, D-LIMONENE, ALPHA-ISOMETHYLIONONE.

### Panel and Study Conditions

A double blind, inter-individual and placebo-controlled clinical evaluation was performed on 44 Caucasian male volunteers (18 years old and more) with white hair.

A first group of 22 volunteers tested the placebo hair lotion and a second group of 22 volunteers tested the hair lotion containing 1% of the hair care active agent of the present invention.

The treatment was applied by massage on the scalp, once a day for four months.

### Results: Reduction of the Proportion of White Hair (Scoring in %)

Pictures of the scalp were taken using a Nikon D7100 in combination with the system Canfield Epiflash®, on the first day of the test and after 4 months of daily application of the product. The hair parting area is defined according to the white hair localization. A blind scoring was performed to evaluate the proportion of while hair in the picture.

The results are shown in the following table:

| | | | | Hair lotion containing 1% of | | |
|---|---|---|---|---|---|---|
| | | | | the hair care active agent | | |
| | Placebo hair lotion | | | of the present invention | | |
| | Mean +/− | Δ% | p | Mean +/− | Δ% | |
| | SEM | mean | value | SEM | mean | p value |
| T₀ | 54.8 ± 5.3 | | | 59.3 ± 4.6 | | ns |
| T_{4M} | 50.2 ± 5 | −8% | p < 0.05 | 49.1 ± 4 | −17% | p < 0.0001 |
| | | | | | | (T0) |
| | | | | | | p < 0.5 |
| | | | | | | (placebo) |

After 4 month of application, a significant reduction of the proportion of white hair by −17%, i.e. 2.1 times more than for the placebo, was observed.

The proportion of white hair was visibly reduced, with a reduction of white hair by −50% for the best respondent (FIG. 9).

### Results: Reduction of White Hair Density (Number/cm²)

Before treatment, an image was taken two days after the shaving of a 1 cm² scalp zone. The instrument used was a Nikon D7100 digital camera in combination with the system Canfield Epiflash® equipped with a contact lens. The contact lens allows flattening hair on the scalp.

A count of white hair was then done with a specific tool of Photoshop on a 0.7 cm² test area (1×0.7 cm) defined on the image. All hairs with a white root within the zone were counted.

The size and the position of the studied area was the same for all evaluation time. In case of offset, the position of the test area was adjusted.

The results are shown in the following table:

| | | | | Hair lotion containing 1% of | | |
|---|---|---|---|---|---|---|
| | | | | the hair care active agent | | |
| | Placebo hair lotion | | | of the present invention | | |
| | Mean +/− | Δ% | | Mean +/− | Δ% | |
| | SEM | mean | p value | SEM | mean | p value |
| T₀ | 41.1 ± 6.1 | | | 49.1 ± 7.3 | | ns |
| T_{4M} | 38.8 ± 4.6 | −5% | ns | 43.4 ± 5.3 | −14% | p < 0.05 (T0) |
| | | | | | | p < 0.1 (placebo) |

The hair lotion containing 1% of the hair care active agent of the present invention significantly decreased the number of white hair per cm², namely by 2.8 times more than the placebo.

After 4 months, the density of white hair was visibly reduced with a decrease of white hair of −55.7 per cm² for the best respondent (FIG. 10).

### EXAMPLE 7: EVALUATION OF MELANIN PRODUCTION BY IN VITRO CO-CULTURE MODEL: COMPARISON

### Introduction

The aim of this study was to compare the effect of the hair care active agent of the present invention, which comprises taxifolin glucoside and N-acetyl tyrosine, with those of L-tyrosine (positive reference), of taxifolin glucoside, and of a mixture of taxifolin glucoside and L-tyrosine. The model used for this comparison was a co-culture of Normal Human Epidermal Keratinocytes (NHEKs) and Normal Human Melanocytes (NHMs).

### Cell Culture

The cell culture was realized with primary cells isolated from human skin biopsies.

Normal Human Keratinocytes (NHKs) were seeded at 60 000 cells per well in 6-wells plates pre-coated with collagen I. 4 hours later, Normal Human Melanocytes (NHMs) were seeded at 60 000 cells per well in the same 6-wells plates. The cells were incubated for 48 hours in complete medium (EpiLife® supplemented with HKGS=Human Kerationcyte Growth Supplement).

After 48 hours of culture, the cells were stimulated for 5 days with the following compositions:
L-tyrosine at 450 μg/mL (Sigma)
Taxifolin glucoside at 0.13 μg/mL
Taxifolin glucoside at 0.13 μg/mL+N-acetyl tyrosine at 15 μg/mL (similar to Example 1)
Taxifolin glucoside at 0.13 μg/mL+L-tyrosine at 15 μg/mL (Sigma)

The compositions were diluted in a basal medium without supplements (EpiLife®). Every 2 days, the treatments were renewed.

### Melanin Extraction and Dosage

After 5 days of treatment, cells were rinsed off with PBS. Then, in each well, 200 μL of a 0.5N NaOH solution was added. The cell lysates were collected in 1.5 mL microtubes with secure caps. In parallel, a standard range of melanin was prepared (100-0 μg/mL). Microtubes of samples and standard range were heated for 1 hour at 80 °C in a dry bath. After the heating, 100 μL of each sample was transferred to a 96-wells plate.

Optical density was measured at 405 nm to determine the melanin content.

### Statistical Analysis

All experiments were conducted at least in triplicate.

A Shapiro-Wilk normality test was realized to evaluate if the data followed the Gaussian Law.

If it was not significant, the data was evaluated with statistical parametric tests (ANOVA followed by Dunnett's multiple comparisons test).

If it was significant, the data was evaluated with statistical non parametric tests (ANOVA of Kruskall-Wallis followed by a Mann-Whitney U test).

### Results

Results are shown in FIG. 11.

The synthesis of melanin was induced by the positive reference (L-tyrosine at 450 μg/ml) by +35% (p<0.001) compared to the untreated condition. This confirms that the co-culture model was responding and able to produce melanin.

Taxifolin glucoside alone, however, was not able to induce synthesis of melanin.

Treatment with a combination of taxifolin glucoside and N-acetyl tyrosine led to an increase in melanin content by +18% (p<0.001) compared to the untreated condition.

The combination of taxifolin glucoside and L-tyrosine, on the other hand, only led to a slight and non-significant increase in melanin content.

There is also a significant difference between the stimulation with taxifolin glucoside and N-acetyl tyrosine and that with taxifolin glucoside and L-tyrosine.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A method of stimulating the proliferation of hair follicle stem cells, stimulating the active melanocytes production, stimulating the melanogenesis, stimulating the recovery of hair pigmentation, reactivating the melanin production in grey hair, activating the antioxidant defenses in hair follicles, protecting the melanocytes against oxidative stress, hair repigmentation, and/or reducing the proportion and/or density of white or grey hair, said method comprising the step of topically a hair care active agent, comprising taxifolin glucoside and N-acetyl tyrosine.

13. The method according to claim 1, wherein at least part of the taxifolin glucoside is taxifolin alpha-D-glucoside.

14. The method according to claim 1, further comprising taxifolin.

15. The method to claim 3, wherein the weight ratio of taxifolin glucoside to taxifolin is from 90:10 to 40:60, more preferably from 70:30 to 50:50, and most preferably about 60:40.

16. The method of claim 1, further comprising glycine and/or epigallocatechin gallate and/or epigallocatechin gallatyl glucoside.

17. The method of claim 1, comprising from 0.01 to 0.50 wt %, more preferably from 0.10 to 0.25 wt %, and most preferably from 0.08 to 0.12 wt %, of taxifolin glucoside.

18. The method according of claim 1, comprising from 1.0 to 30 wt %, more preferably from 10 to 20 wt %, and most preferably from 13.5 to 16.5 wt %, of N-acetyl tyrosine.

19. The method according of claim 1, wherein the weight ratio of taxifolin glucoside to N-acetyl tyrosine is from 1: 5,100 to 1:2, more preferably from 1:340 to 1:70, and most preferably from 1:200 to 1:150.

20. The method according to claim 2, further comprising taxifolin.
